(19) United States
(12) Patent Application Publication
(10) Pub. No.: US 2022/0330935 A1
(43) Pub. Date: Oct. 20, 2022

(54) TISSUE FASTENING

(71) Applicant: Children's Medical Center Corporation, Boston, MA (US)

(72) Inventors: Alexander Yang, Boston, MA (US); Robert W. Crum, Brookline, MA (US); Kyle C. Wu, Boston, MA (US); Heung Bae Kim, Weston, MA (US); Brianna L. Slatnick, Cambridge, MA (US); Farokh R. Demehri, Brookline, MA (US)

(21) Appl. No.: 17/855,719

(22) Filed: Jun. 30, 2022

Related U.S. Application Data
(63) Continuation of application No. PCT/US21/14352, filed on Jan. 21, 2021.
(60) Provisional application No. 62/963,740, filed on Jan. 21, 2020.

(51) Int. Cl. A61B 17/04

(52) U.S. Cl. CPC A61B 17/0469; A61B 2017/0458; A61B 2017/0417; A61B 2017/0496

(57) ABSTRACT

The fastener assemblies, systems, and methods of the present disclosure are generally directed to a first T-fastener and a second T-fastener securable to one another through the use of a suture extending therebetween and deliverable to an anatomical location using a minimally invasive technique. For example, a cannula of a needle assembly may be percutaneously deliverable to a treatment site and, through actuation of a hub of the needle assembly, the first T-fastener and the second T-fastener may be deliverable relative to biological tissue to be fastened at the treatment site. The suture may intracorporeally fasten the first T-fastener and the second T-fastener relative to one another for robust securement biological tissue therebetween. As compared to securement using external fixation, the fastener assemblies, systems, and methods of the present disclosure may facilitate intracorporeally fastening tissue while reducing or eliminating certain requirements associated with postoperative care.

### RELATED APPLICATIONS

This application is a bypass continuation that claims priority to International Patent Application No. PCT/US21/14352 filed on Jan. 21, 2021, which claims priority to U.S. Prov. App. No. 62/963,740 filed on Jan. 21, 2020, where the entire content of each of the foregoing is hereby incorporated by reference.

### TECHNICAL FIELD

The disclosure generally relates to fastening tissue and, more particularly, to T-fasteners for securing biological tissue therebetween.

### BACKGROUND

In certain medical procedures, it is desirable to secure biological tissue in a particular anatomical location and/or relative to an implant. While various fasteners are used to achieve such securement, locking these fasteners in place creates certain challenges.

As an example, in procedures such as gastrostomy tube (G-Tube) placement, fasteners are used to secure the stomach wall to the abdominal wall of a subject. This may be achieved by percutaneously inserting a T-fastener through both the anterior abdominal wall and the stomach wall and then retracting the T-fastener to create the desired apposition of the wall layers. A locking disc may then be secured to the T-fastener on the surface of the skin to hold the T-fastener tightly against the internal surface of the stomach wall. The locking disc on the surface of the skin can, however, complicate postoperative care. For example, to the extent a post-operative visit is required for removal of the locking disc, the use of a locking disc may be less desirable for patient populations with limited access to healthcare. Further, the locking disc on the surface of the skin can cause discomfort, pressure ulcers on the skin, or other complications. Still further, because the locking disc is secured to the implanted T-fastener via an open incision, prophylactic measures are often required to reduce the likelihood of infection at the site of the incision.

As another example, in hernia repair procedures, a synthetic mesh is secured to the anterior abdominal wall of a patient using laparoscopically delivered tacks. While these tacks have screw-like features that engage with the abdominal wall, there is nothing on the other side of the abdominal wall to hold these tacks in place. Thus, these tacks can become dislodged. In the event of such dislodgement, the placement of the synthetic mesh may become compromised and, in some cases, additional surgical intervention may be required.

There remains a need for fastening techniques that are executable intracorporeally while providing robust fixation and reduced postoperative complications.

### SUMMARY

The fastener assemblies, systems, and methods of the present disclosure are generally directed to a first T-fastener and a second T-fastener securable to one another through the use of a suture extending therebetween and deliverable to an anatomical location using a minimally invasive technique. For example, a cannula of a needle assembly may be percutaneously deliverable to a treatment site and, through actuation of a hub of the needle assembly, the first T-fastener and the second T-fastener may be deliverable relative to biological tissue to be fastened at the treatment site. The suture may intracorporeally fasten the first T-fastener and the second T-fastener relative to one another for robust securement of biological tissue therebetween. As compared to securement using external fixation, the fastener assemblies, systems, and methods of the present disclosure may facilitate intracorporeally fastening tissue while reducing or eliminating certain requirements associated with postoperative care.

According to one aspect, a fastener assembly for intracorporeal tissue apposition may include a first T-fastener having a first elongate body defining a plurality of first openings, a second T-fastener having a second elongate body defining a plurality of second openings, and a suture having a first end portion, a second end portion, and a length therebetween, the length of the suture extending through the plurality of first openings and the plurality of second openings with the second T-fastener, along the length of the suture, between the first T-fastener and each of the first end portion and the second end portion of the suture.

In some implementations, at least the second T-fastener may be movable along the length of the suture in a direction toward the first T-fastener. Additionally, or alternatively, the first T-fastener may be secured in a fixed position along the length of the suture.

In certain implementations, at least a portion of one or more of the first elongate body of the first T-fastener, the second elongate body of the second T-fastener, or the suture may be bioabsorbable.

In some implementations, each first opening of the plurality of first openings may define a respective first center axis, each second opening of the plurality of second openings may define a respective second center axis, and, with tension in the length of the suture, each first center axis associated with the plurality of first openings of the first T-fastener may be movable into substantial coaxial alignment with a respective second center axis associated with the plurality of second openings of the second T-fastener. As an example, the first center axes defined by the plurality of first openings may be substantially parallel to one another and spaced relative to one another by a first distance, and the second center axes defined by the plurality of second openings may be substantially parallel to one another and spaced relative to one another by a second distance about equal to the first distance. Further, or instead, the first center axes may define a first plane substantially perpendicular to a first longitudinal axis defined by the first elongate body of the first T-fastener. Additionally, or alternatively, the second center axes may define a second plane substantially perpendicular to a second longitudinal axis defined by the second elongate body of the second T-fastener. In some instances, the second elongate body of the second T-fastener may have a maximum radial dimension along the second plane and, in each direction away from the second plane along the second longitudinal axis, the second elongate body of the second T-fastener may have a decreasing radial dimension.

In certain implementations, the first elongate body of the first T-fastener may define a pair of elongate grooves extending longitudinally along a longitudinal dimension of the first elongate body, and a portion of the length of the suture may be positionable in the pair of elongate grooves.

In some implementations, the suture further may include a plurality of barbs, each barb of the plurality of barbs may extend radially from the length of the suture, and at least one of the first end portion of the suture or the second end portion of the suture may be movable through the plurality of second openings of the second T-fastener in a first direction to move at least one barb of the plurality of barbs through at least one opening of the plurality of second openings.

According to another aspect, a system for intracorporeal delivery of a fastener assembly may include a first T-fastener having a first elongate body, a second T-fastener having a second elongate body, and a suture having a first end portion, a second end portion, and a length therebetween, the length of the suture extending through a plurality of first openings defined by the first elongate body and through a second plurality of openings defined by the second elongate body, and a needle assembly including a hub and a cannula, the cannula having a proximal section and a distal section, the hub coupled to the proximal section of the cannula, the cannula defining a lumen extending from the proximal section to the distal section, and each of the first T-fastener, the second T-fastener, and at least a portion of the length of the suture disposed in the lumen.

In some implementations, the first elongate body may define a first longitudinal axis, the second elongate body may define a second longitudinal axis, and the cannula may define a cannula center axis substantially aligned with the first longitudinal axis and the second longitudinal axis. For example, with the second longitudinal axis of the second T-fastener substantially aligned with the cannula center axis in the lumen, the first T-fastener may be positionable beyond the distal section of the cannula with the first longitudinal axis of the first T-fastener intersecting the second longitudinal axis of the second T-fastener.

In certain implementations, the first end portion and the second end portion of the suture may be disposed outside of the needle assembly. For example, the cannula may define a slit extending from the proximal section to the distal section, the slit may have a cross-sectional dimension larger than a radial dimension of the suture, and the length of the suture extends through the slit with the first end portion and the second end portion of the suture outside of the cannula.

In some implementations, the system may further include a plunger in contact with the second T-fastener, the plunger may be depressible in a first discrete movement in a distal direction in the cannula, and the first discrete movement of the plunger in the distal direction may move the first T-fastener, the second T-fastener, and at least a portion of the length of the suture in the distal direction along the lumen. For example, the plunger may be depressible in a second discrete movement in the distal direction in the cannula, the first discrete movement of the plunger in the distal direction delivering the first T-fastener from the distal section of the cannula, and the second discrete movement of the plunger in the distal direction delivering the second T-fastener from the distal section of the cannula. Additionally, or alternatively, between the first discrete movement and the second discrete movement in the distal direction in the cannula, the plunger may be movable in a proximal direction in the cannula.

According to yet another aspect, a method for intracorporeal delivery of a fastener assembly to a mammal may include moving a distal section of a cannula at least partially through biological tissue to a first anatomical location, with the distal section of the cannula at the first anatomical location, delivering a first T-fastener from the cannula to the first anatomical location, with the first T-fastener at the first anatomical location, retracting the distal section of the cannula through the biological tissue to a second anatomical location, delivering a second T-fastener from the distal section of the cannula to the second anatomical location, and moving the first T-fastener and the second T-fastener toward one another along a suture extending through each of the first T-fastener and the second T-fastener, the movement of the first T-fastener and the second T-fastener securing the biological tissue therebetween.

### BRIEF DESCRIPTION OF THE DRAWINGS

The foregoing and other objects, features, and advantages of the devices, systems, and methods described herein will be apparent from the following description of particular embodiments thereof, as shown in the accompanying figures. The figures are not necessarily to scale, emphasis instead being placed upon describing the principles of the devices, systems, and methods described herein.

FIG. 1A is a perspective view of a system including a needle assembly and a fastener assembly.

FIG. 1B is a perspective view of the system of FIG. 1A, shown in partial cross-section with a portion of a hub and a portion of a cannula of the needle assembly removed along the line 1B-1B in FIG. 1A.

FIG. 1C is a partially exploded, perspective view of the partial cross-section of the system shown in FIG. 1B.

FIG. 1D is a perspective view of a partial cross-section of the hub of FIG. 1B.

FIG. 2A is a perspective view of a fastener assembly of the system of FIG. 1A.

FIG. 2B is a perspective view of a first T-fastener of the fastener assembly of FIG. 2A.

FIG. 2C is a perspective view of a second T-fastener of the fastener assembly of FIG. 2C.

FIG. 2D is a side, cross-sectional view of the first T-fastener of the fastener assembly of FIG. 2A, the cross-section taken along the line 2D-2D in FIG. 2B.

FIG. 2E is a side, cross-sectional view of the second T-fastener of the fastener assembly of FIG. 2A, the cross-section taken along the line 2E-2E in FIG. 2C.

FIG. 3A is the first schematic representation of a temporal sequence of activation of the needle assembly of FIG. 1A to deliver the fastener assembly of FIG. 2A, the temporal sequence is shown according to the partial cross-section of FIG. 1B.

FIG. 3B is the second schematic representation of a temporal sequence of activation of the needle assembly of FIG. 1A to deliver the fastener assembly of FIG. 2A, the temporal sequence is shown according to the partial cross-section of FIG. 1B.

FIG. 3C is the third schematic representation of a temporal sequence of activation of the needle assembly of FIG. 1A to deliver the fastener assembly of FIG. 2A, the temporal sequence is shown according to the partial cross-section of FIG. 1B.

FIG. 3D is the fourth schematic representation of a temporal sequence of activation of the needle assembly of FIG. 1A to deliver the fastener assembly of FIG. 2A, the temporal sequence is shown according to the partial cross-section of FIG. 1B.

FIG. 3E is the fifth schematic representation of a temporal sequence of activation of the needle assembly of FIG. 1A to deliver the fastener assembly of FIG. 2A, the temporal sequence is shown according to the partial cross-section of FIG. 1B.

FIG. 4A is the first schematic representation of a temporal sequence of an exemplary method of intracorporeal delivery of a fastener assembly to a mammal.

FIG. 4B is the second schematic representation of a temporal sequence of an exemplary method of intracorporeal delivery of a fastener assembly to a mammal.

FIG. 4C is the third schematic representation of a temporal sequence of an exemplary method of intracorporeal delivery of a fastener assembly to a mammal.

FIG. 4D is the fourth schematic representation of a temporal sequence of an exemplary method of intracorporeal delivery of a fastener assembly to a mammal.

FIG. 4E is the fifth schematic representation of a temporal sequence of an exemplary method of intracorporeal delivery of a fastener assembly to a mammal.

FIG. 4F is the sixth schematic representation of a temporal sequence of an exemplary method of intracorporeal delivery of a fastener assembly to a mammal.

FIG. 4G is the seventh schematic representation of a temporal sequence of an exemplary method of intracorporeal delivery of a fastener assembly to a mammal.

FIG. 5A is a schematic representation of a system including a needle assembly, the needle assembly including a hub releasably securable to a plurality of cannulas.

FIG. 5B is a perspective view of the system of FIG. 5A, shown in partial cross-section with a portion of a hub and a portion of a cannula of the needle assembly removed.

FIG. 6 is a perspective view of a cannula defining a slit.

FIG. 7 is a cross-sectional side view of a fastener assembly including a suture having barbs.

FIG. 8 is a cross-sectional side view of a fastener assembly including a suture having barbs engageable with a fastener.

Like reference symbols in the various drawings indicate like elements.

### DETAILED DESCRIPTION

The embodiments will now be described more fully hereinafter with reference to the accompanying figures, in which certain embodiments are shown. The foregoing may, however, be embodied in many different forms and should not be construed as limited to the embodiments shown herein.

All documents mentioned herein are hereby incorporated by reference in their entirety. References to items in the singular should be understood to include items in the plural, and vice versa, unless explicitly stated otherwise or clear from the context. Grammatical conjunctions are intended to express any and all disjunctive and conjunctive combinations of conjoined clauses, sentences, words, and the like, unless otherwise stated or clear from the context. Thus, unless otherwise indicated or made clear from the context, the term “or” should generally be understood to mean “and/or,” and the term “and” should generally be understood to mean “and/or.”

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended or stated purpose. Ranges of values and/or numeric values are provided herein as examples only, and do not constitute a limitation on the scope of the described embodiments. The use of any and all examples or exemplary language (“e.g.,” “such as,” or the like) provided herein, is intended to better describe the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” and the like, are words of convenience and are not to be construed as limiting terms, unless specifically stated.

As used herein, the term “clinician” shall be generally understood to refer to a care provider interacting with any portion of the devices and/or systems described herein in the course of preparing for or carrying out a medical procedure on a subject. Thus, for example, the term clinician is intended to include a physician, a nurse, or other medical professionals. Further, the term clinician may include support personnel assisting a medical professional in preparing for or carrying out a medical procedure.

Also, as used herein, the term “subject” shall be generally understood to be a mammal. Thus, the term subject shall be understood to include humans, as well as any other mammals treatable according to the techniques described herein. Stated differently, unless otherwise specified or made clear from the context, the devices, systems, and methods of the present disclosure shall be understood to be applicable to medical treatment of humans, veterinary treatment of other mammals, or teaching/research environments using mammalian tissue, such as tissue harvested from a cadaver.

For the sake of clarity and economy of explanation, the devices, systems, and methods of the present disclosure are generally described with respect to securing the stomach wall in apposition to the abdominal wall as part of a G-tube placement procedure in a subject. Unless otherwise specified, or made clear from the context, however, it should be more generally understood that the devices, systems, and methods of the present disclosure may be used in any medical procedure in which it may be useful or necessary to fasten biological tissue, such as in instances in which clinical outcomes of such procedures may benefit from the reduction or elimination of external fixation devices and/or in instances in which anatomical constraints make the use of external fixation devices impractical or unfeasible. Thus, by way of example, the devices, systems, and methods of the present disclosure may be used to fasten any one or more layers of biological tissue in a fixed orientation (e.g., in apposition) to one another in any of various different types of procedures. Further, or instead, the devices, systems, and methods of the present disclosure may be used to fasten an implant to biological tissue during the course of any one or more of various different procedures, such as fastening a synthetic mesh to the anterior abdominal wall of the subject in the course of a hernia repair procedure.

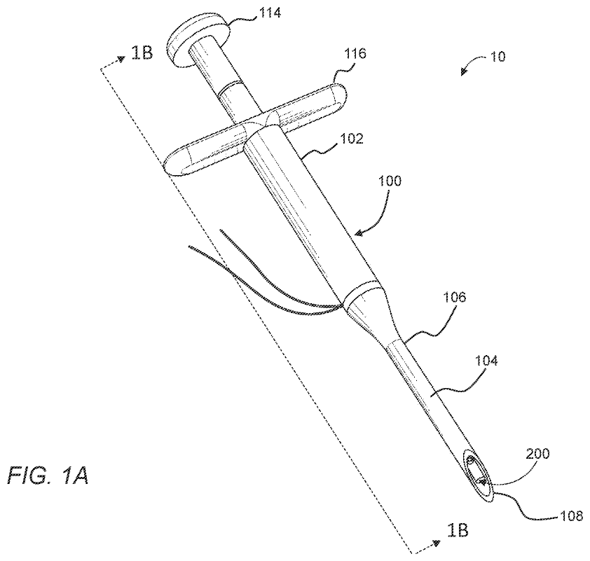

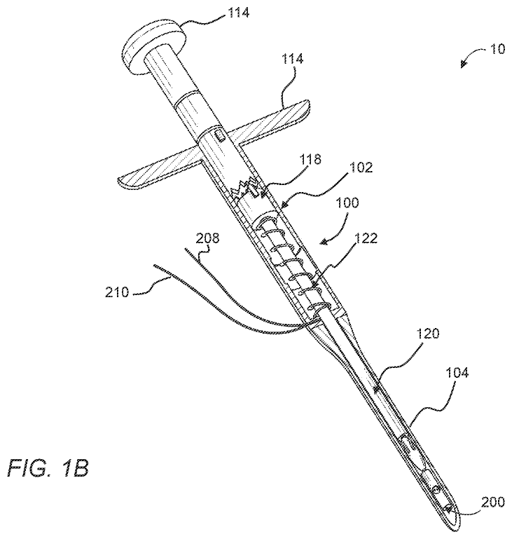

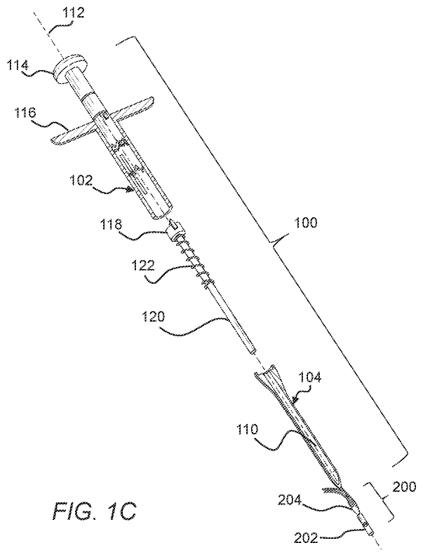

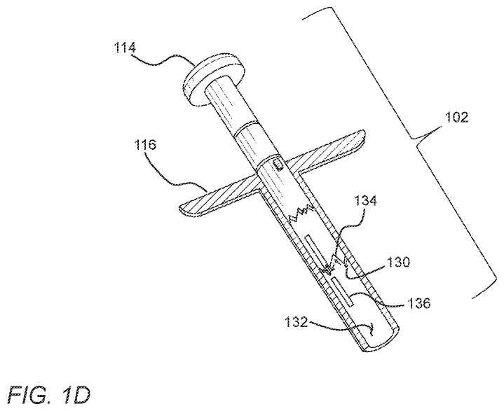

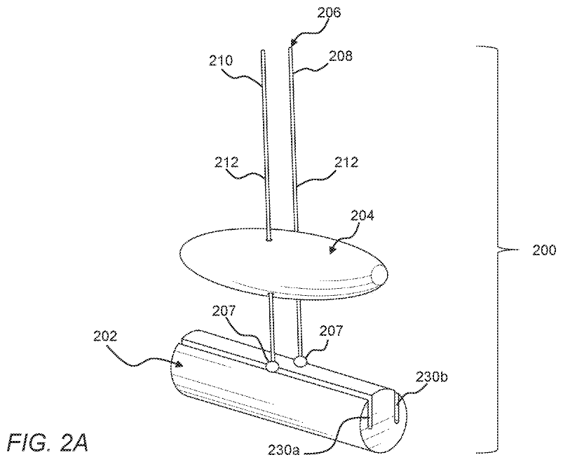

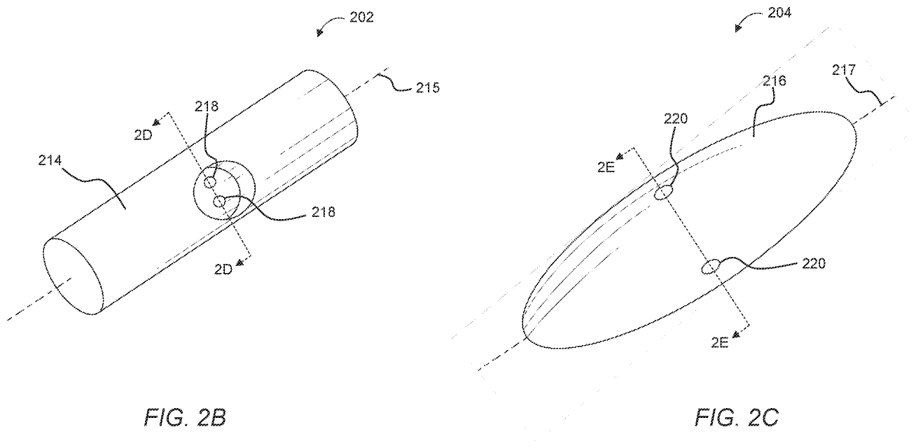

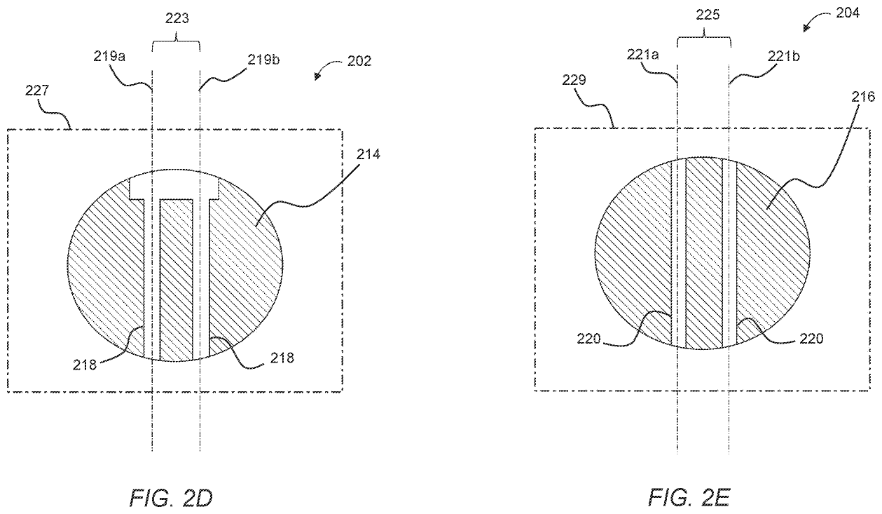

Referring now to FIGS. 1A, 1B, 1C, 1D, 2A, 2B, 2C, 2D, and 2E, a system 10 may include a needle assembly 100 and a fastener assembly 200. As described in greater detail below, at least a portion of the fastener assembly 200 may be disposed within the needle assembly 100 such that the fastener assembly 200 is percutaneously deliverable to a treatment site via the needle assembly 100. As compared to accessing a treatment site through the use of a surgical incision, the system 10 may provide less invasive access to the treatment site, in turn facilitating faster postoperative recovery. As also described in greater detail below, the fastener assembly 200 may be intracorporeally secured to hold biological tissue (e.g., one or more layers of biological tissue alone or in combination with an implant) in place as appropriate for a given procedure. As should be generally appreciated, such intracorporeal securement may have significant advantages relative to external tissue securement techniques, ultimately reducing healthcare resources required to achieve comparable or improved clinical outcomes for certain medical procedures. For example, as compared to laparoscopic suturing (e.g., as part of G-tube placement or hernia repair), percutaneously delivering the fastener assembly 200 to a treatment site and intracorporeally securing the fastener assembly 200 to secure the abdominal wall to the stomach wall may be less technically demanding of the clinician and, further or instead, may be less time-consuming. Additionally, or alternatively, as compared to using external fixation to hold one or more fasteners in place at a treatment site (e.g., as part of G-tube placement), the intracorporeal securement of the fastener assembly 200 may reduce the likelihood of infection, discomfort, skin conditions, and, more generally, the overall demands of post-operative care. Still further, or instead, as compared to using laparoscopically delivered tacks to secure an implant in place (e.g., as part of hernia repair), the fastener assembly 200 may offer securement that is more robust in response to typical postoperative conditions.

Referring now specifically to FIGS. 2A, 2B, 2C, 2D, and 2E, the fastener assembly 200 may include a first T-fastener 202, a second T-fastener 204, and a suture 206. The suture 206 may have a first end portion 208, a second end portion 210, and a length 212 therebetween. In general, the first T-fastener 202 and the second T-fastener 204 may be supported along the length 212 of the suture 206, between the first end portion 208 and the second end portion 210 of the suture 206. As this suggests, the term “length” in this context shall be understood to refer to a portion of the suture 206 between the first end portion 208 and the second end portion 210 and, unless otherwise specified or made clear from the context, is not necessarily intended to refer to a measured distance, nor is it intended to require a particular shape or orientation. Thus, in general, at least the second T-fastener 204 may be movable (e.g., pushable by a clinician) along the length 212 of the suture 206, in a direction toward the first T-fastener 202, to secure biological tissue between the second T-fastener 204 and the first T-fastener 202. In certain instances, the first T-fastener 202 may be secured in a fixed position along the length 212 of the suture 206 (e.g., by one or more restrictions 207 of the suture 206, such as knots, an adhesive, or a combination thereof) to facilitate accurate placement of the fastener assembly 200 at the treatment site. For example, with the first T-fastener 202 secured in a fixed position along the length 212 of the suture 206, the first T-fastener 202 may be less likely to undergo unintentional movement in response to forces required to move the first T-fastener 202 and the second T-fastener 204 toward one another to secure biological tissue therebetween.

In certain implementations, the suture 206 may be generally in the shape of a filament (e.g., monofilament or braided) along the length 212 of the suture 206. For example, in the absence of tension applied to the first end portion 208 and the second end portion 210, the suture 206 may be flexible along the length 212 to facilitate placing the first T-fastener 202 and the second T-fastener 204 on the suture 206 to form the fastener assembly 200 and, additionally or alternatively, to facilitate moving the fastener assembly 200 to the treatment site. Further, or instead, the suture 206 may be pulled taut, along the length 212, through tension applied to the first end portion 208 and the second end portion 210. In this taut state, the length 212 of the suture 206 may restrict movement of the first T-fastener 202 and the second T-fastener 204 such that at least one of the first T-fastener 202 and the second T-fastener 204 may be pushed along the length 212 of the suture 206 in the taut state. It should be generally appreciated that the ability to position the first T-fastener 202 and the second T-fastener 204 relative to one another using only a single degree of freedom defined by the suture 206 in the taut state may facilitate repeated and accurate alignment of the first T-fastener 202 and the second T-fastener 204 relative to one another at the treatment site.

The suture 206 may be formed of any one or more of various different biocompatible materials formable into an elongate form factor and having sufficient strength to resist tension (e.g., without significant plastic deformation or, at least, without breaking) associated with implantation of the fastener assembly 200 and securing biological tissue between the first T-fastener 202 and the second T-fastener 204. In certain instances, the suture 206 may be formed of one or more bioabsorbable materials. In this context, bioabsorbable materials shall be understood to include any one or more materials that, over a period of time (e.g., over the course of days, weeks, or another period of time longer than typical healing times for a given procedure), may be absorbed into living tissue of the subject or otherwise biodegradable in an implanted environment. As may be appreciated, such bioabsorbable materials may reduce or eliminate the need for postoperative intervention (e.g., surgical intervention) to remove the fastener assembly 200, thus reducing time, cost, and complexity associated with achieving positive clinical outcomes. Thus, in general, the suture 206 may include any one or more of various different suture materials known in the art and, more specifically, may include one or more of nylon, polypropylene, silk, polyester, or a combination thereof.

In general, the first T-fastener 202 and the second T-fastener 204 may each have orientation-specific size profiles. That is, as described in greater detail below, the first T-fastener 202 and the second T-fastener 204 may have a first orientation relative to one another and relative to one or more components of the needle assembly to facilitate percutaneous delivery of the fastener assembly 200 to the treatment site. As also described in greater detail below, the first T-fastener 202 and the second T-fastener 204 may have a second orientation, different from the first orientation, useful for resisting unintended retraction of one or both of the first T-fastener 202 or the second T-fastener 204 through holes made in biological tissue during percutaneous delivery of the fastener assembly 200 to the treatment site.

As an example of an orientation-specific profile, the first T-fastener 202 may have a first elongate body 214 defining a first longitudinal axis 215, and the second T-fastener 204 may have a second elongate body 216 defining a second longitudinal axis 217. As used herein, the term “elongate body” shall be understood to include any three-dimensional shape having a length dimension greater than any other dimension of the respective shape. In some instances, one or both of the first elongate body 214 or the second elongate body 216 may be substantially revolute shapes. As used in this context, a substantially revolute shape shall be understood to include a revolute shape formed about the respective longitudinal axis (e.g., a longer or longest axis) associated with the given fastener, with deviations from a geometric revolute shape to accommodate certain surface features such as holes, recesses, texture, or a combination thereof. Such substantially revolute shapes may be, for example, atraumatic such that the one or both of the first elongate body 214 or the second elongate body 216 lacks sharp edges, corners, or protrusions likely to pierce biological tissue or an implant during positioning of the first elongate body 214 or the second elongate body 216, as the case may be. Further, or instead, such substantially revolute shapes may facilitate fabrication according to controlled dimensional tolerances.

In certain implementations, the first elongate body 214 of the first T-fastener 202 may define a plurality of first openings 218, and the second elongate body 216 of the second T-fastener 204 may define a plurality of second openings 220. The length 212 of the suture 206 may extend through the plurality of first openings 218 of the first T-fastener 202 and through the plurality of second openings 220 of the second T-fastener 204. More specifically, the length 212 of the suture 206 may extend through the plurality of first openings 218 of the first T-fastener 202 and through the plurality of second openings 220 such that the second T-fastener 204, along the length 212 of the suture 206, is between the first T-fastener 202 and each of the first end portion 208 and the second end portion 210 of the suture 206. Stated differently, the fastener assembly 200 may be formed by first threading the first end portion 208 and the second end portion 210 of the suture 206 through the plurality of first openings 218 of the first T-fastener 202 before threading the first end portion 208 and the second end portion 210 of the suture through the plurality of second openings 220 of the second T-fastener 204. In this context, it will be appreciated that the description is geometric rather than chronological. That is, the suture may be threaded through the openings in any order that results in the threading configuration illustrated, e.g., in FIG. 2A. Such positioning of the first T-fastener 202 relative to the second T-fastener 204 along the length 212 of the suture may facilitate percutaneously delivering the first T-fastener 202 and the second T-fastener 204 to a treatment site in a predetermined orientation relative to one another, with the first T-fastener 202 deliverable to the treatment site prior to delivery of the second T-fastener to the treatment site, as described in greater detail below. It will also be appreciated that any similarly functional combination of sutures may be used without departing from the scope of this disclosure. For example, a first suture may be coupled in a fixed manner to the first T-fastener 202, e.g., using a knot, adhesive, or other attachment technique, and then threaded through a hole in the second T-fastener 204. A second suture may similarly be attached to the first T-fastener 202 and threaded through a different hole in the second T-fastener 204. More generally, a system may use any number and arrangement of sutures coupled to the T-fasteners in a manner that facilitates deployment and use of the T-fasteners for the surgical procedures described herein.

In general, the plurality of first openings 218 and the plurality of second openings 220 may be any one or more of various different types of openings suitable for receiving the length 212 of the suture 206 such that the first T-fastener 202 and the second T-fastener 204 may be supported along the length 212 of the suture 206. Thus, for example, the plurality of first openings 218 and the plurality of second openings 220 may each be through-holes extending through the first elongate body 214 of the first T-fastener 202 and through the second elongate body 216 of the second T-fastener 204, respectively. As compared to other types of openings, such through-holes may facilitate threading the suture 206 through the first T-fastener 202 and the second T-fastener 204 to form the fastener assembly 200 by hand or, at least, without the use of specialized tools. Further, or instead, each of the plurality of first openings 218 and the plurality of second openings 220 may be a respective pair of openings to facilitate threading the suture 206 through the first T-fastener 202 and the second T-fastener 204. It should be appreciated, however, that each of the plurality of first openings 218 or the plurality of second openings 220 may include additional openings, and each set of openings may include a different number of openings than the other set of openings.

In some implementations, each first opening 218 of the plurality of first openings 218 may be substantially alignable with respective second opening 220 of the plurality of second openings 220 as the first T-fastener 202 and the second T-fastener 204 are pulled toward one another along the length 212 of the suture 206 in the taut state to secure biological tissue with the fastener assembly 200. As an example, each of the first openings 218 in the plurality of first openings 218 may define a respective first center axis 219a,b extending through the first elongate body 214 of the first T-fastener 202. Continuing with this example, each of the second openings 220 in the plurality of second openings 220 may define a respective second center axis 221a,b extending through the second elongate body 216 of the second T-fastener 204. With tension in the length 212 of the suture 206, each first center axis 219a,b associated with the plurality of first openings 218 may be movable into substantial coaxial alignment (e.g., within ±15 degrees) with a respective second center axis 221a,b associated with the plurality of second openings of the second T-fastener. That is, the first center axis 219a and the second center axis 221a may be movable into substantially coaxial alignment with one another and, similarly, the first center axis 219b and the second center axis 221b may be movable into substantially coaxial alignment with one another. As compared to openings that are not alignable with one another, such alignability may, for example, reduce the likelihood of tangling the suture 206 as the first T-fastener 202 and the second T-fastener 204 are drawn toward one another. Further, or instead, as compared to openings that are not alignable with one another, alignability of the plurality of first openings 218 to the plurality of second openings 220 may reduce the likelihood of creating undesirably high tension in the suture 206 as the first T-fastener 202 and the second T-fastener 204 are pulled toward one another at the treatment site to secure biological tissue therebetween.

In certain instances, the plurality of first openings 218 may be disposed along the first elongate body 214 of the first T-fastener 202 and oriented relative to one another based on, among other things, considerations associated with forces exerted by the first T-fastener 202 on the length 212 of the suture 206 in tension. Additionally, or alternatively, the plurality of second openings 220 may be placed along the second elongate body 216 and oriented relative to one another to according to analogous considerations associated with second T-fastener 204 on the length 212 of the suture 206 in tension. For example, the first center axes 219a,b, may be substantially parallel to one another (e.g., within ±15 degrees) and spaced relative to one another by a first distance 223 such that the plurality of first openings 218 are substantially parallel to one another. Continuing with this example, the second center axes 221a,b defined by the plurality of second openings 220 may be substantially parallel to one another (e.g., within ±15 degrees) and spaced relative to one another by a second distance 225 such that the plurality of second openings 220 are substantially parallel to one another. As compared to a non-parallel orientation, orienting the plurality of first openings 218 substantially parallel to one another and/or orienting the plurality of second openings 220 substantially parallel to one another may reduce frictional force on the length 212 of the suture 206 as the first T-fastener 202 or the second T-fastener 204, as the case may be, is moved along the length 212 of the suture 206 in tension. As a more specific example, the second distance 225 between second center axes 221a,b may be substantially equal (allowing for dimensional tolerances) to the first distance 223 between the first center axes 219a,b, which may be particularly useful for reducing the likelihood of excessive frictional forces exerted on the length 212 of the suture 206 as the first T-fastener 202 and the second T-fastener 204 are moved along the length 212 of the suture 206 in tension.

Further, or instead, the plurality of first openings 218 may be disposed along the first elongate body 214 of the first T-fastener 202 and the plurality of second openings 220 may be placed along the second elongate body 216 of the second T-fastener 204 according to considerations related to positioning the first T-fastener 202 and the second T-fastener 204, as the case may be, relative to biological tissue at the treatment site. For example, in some implementations, it may be desirable to position the plurality of first openings 218 along the first elongate body 214 such that, with the length 212 of the suture 206 in tension, the first T-fastener 202 may have a tendency to pivot (e.g., through contact with biological tissue) relative to a direction of insertion of the needle assembly 100 (FIG. 1A) to the treatment site. As described in greater detail below, such pivoting may self-align the first elongate body 214 of the first T-fastener 202 relative to biological tissue to restrict movement of the first T-fastener 202 back along the direction of insertion of the needle assembly 100 (FIG. 1A) following retraction of the needle assembly 100 back through the biological tissue. Analogous considerations should be understood to be applicable to placement of the plurality of second openings 220 along the second elongate body 216 of the second T-fastener 204.

By way of example, the first center axes 219a,b defined by the plurality of first openings 218 may define a first plane 227 substantially perpendicular to the first longitudinal axis 215 defined by the first elongate body 214 of the first T-fastener 202. Continuing with this example, with tension applied along the length 212 of the suture 206, the first longitudinal axis 215—and, thus, the first elongate body 216—may pivot about a point of intersection between the first longitudinal axis 215 and the first plane 227. Through such pivoting, as the first elongate body 214 comes into contact with biological tissue at the treatment site while tension is applied along the length 212 of the suture 206, the first elongate body 214 may substantially self-align to an orientation away from a direction of insertion of the needle assembly 100 (FIG. 1A). That is, with the plurality of first openings 218 so defined relative to the first longitudinal axis 215, the first elongate body 214 of the first T-fastener 202 may be, once delivered from the needle assembly 100, biased toward an orientation that restricts undesirable movement of the first T-fastener 202 at the treatment site while the suture 206 is being secured to hold the first T-fastener 202 and the second T-fastener 204 in place relative to one another.

As an additional or alternative example, the second center axes 221a,b may define a second plane 229 substantially perpendicular to the second longitudinal axis 217 defined by the second elongate body 216 of the second T-fastener 204. Thus, orientation of the second longitudinal axis 217 may tend to pivot in a manner analogous to the pivoting described above with respect to the first longitudinal axis 215. Accordingly, the second elongate body 216 may substantially self-align to an orientation away from the direction of insertion of the needle assembly 100 (FIG. 1A) to restrict undesirable movement of the second T-fastener 204 at the treatment site while the suture 206 is being secured to hold the first T-fastener 202 and the second T-fastener 204 in place relative to one another.

In general, the first T-fastener 202 and the second T-fastener 204 may be formed of any one or more of various different biocompatible materials. For example, in some instances, one or both of the first T-fastener 202 or the second T-fastener 204 may be formed of a bioabsorable material. Continuing with this example, the bioabsorbable material may be absorbable at the treatment site over a period that is longer than a typical recovery time associated with a given procedure such that one or both of the first T-fastener 202 or the second T-fastener 204, as the case may be, may be absorbed or otherwise undergo benign degradation at the treatment site after recovery has occurred at the treatment site. In certain instances, one or both of the first T-fastener 202 or the second T-fastener 204 may be a bioabsorbable polymer, such as polydioxanone, that may be moldable into a suitable initial shape, withstands typical fastening forces during the recovery period, and may be absorbed following a typical recovery period. As compared to materials that are not bioabsorbable, it should be generally appreciated that forming one or more of the first T-fastener 202 or the second T-fastener 204 of a bioabsorbable material may reduce the need for postoperative intervention to remove the first T-fastener 202 or the second T-fastener 204, as the case may be.

Referring now to FIGS. 1A, 1B, 1C, 1D, 2A, 2B, 2C, 2D, and 2E, while the first T-fastener 202 and the second T-fastener 204 have been described with respect to features associated with interaction between one or both of the first T-fastener 202 or the second T-fastener 204 with biological tissue at the treatment site, one or both of the first T-fastener 202 or the second T-fastener 204 may include additional or alternative features useful for facilitating delivery from the needle assembly 100. For example, the second T-fastener 204 may have a maximum radial dimension along the second plane 229. Further, or instead, in each direction away from the second plane 229 along the second longitudinal axis 217, the second elongate body 216 may have a decreasing (e.g., gradually tapering) radial dimension. Stated differently, the second elongate body 216 may be shaped such that each end portion of the second elongate body 216 has a smaller radial dimension than a middle portion of the second elongate body 216. In general, smaller radial dimensions along each end portion of the second elongate body 216 may reduce the likelihood of damage to the second elongate body 216 by the needle assembly 100 as the second T-fastener 204 is delivered from the needle assembly 100. That is, as described in greater detail below, delivery of the second T-fastener 204 from the needle assembly 100 may include tilting the needle assembly 100, and smaller radial dimensions along each end portion of the second elongate body 216 may reduce the likelihood of inadvertent contact between the second T-fastener 204 and the titled needle assembly 100.

Further, or instead, one or more of the first T-fastener 202 or the second T-fastener 204 may include features useful for storing the fastener assembly 200 in the needle assembly 100 prior to use and, ultimately, during percutaneous delivery of the fastener assembly 200 to the treatment site. For example, to facilitate percutaneous delivery of the fastener assembly 200 via the needle assembly 100, one or more of the first T-fastener 202 or the second T-fastener 204 may include one or more features useful for accommodating the length 212 of the suture 206 while the fastener assembly 200 is stored in the needle assembly 100. As a more specific example, the first elongate body 214 of the first T-fastener 202 may define a pair of elongate grooves 230a,b, with the elongate groove 230a and the elongate groove 230b each extending longitudinally along the longitudinal dimension of the first elongate body 214. Continuing with this example, with the fastener assembly 200 stored in the needle assembly 100, at least a portion of the length 212 of the suture 206 may be positionable in the pair of elongate grooves 230a,b. As compared to storing the length 212 of the suture 206 next to an external surface of the first T-fastener 202, positioning at least a portion of the length 212 of the suture 206 in the pair of elongate grooves 230a,b may reduce the likelihood of crushing or otherwise damaging the length 212 of the suture 206 while the fastener assembly 200 is stored in the needle assembly 100.

Referring now specifically to FIGS. 1A, 1B, 1C, and 1D, the needle assembly 100 may include a hub 102 and a cannula 104. The cannula 104 may have a proximal section 106 and a distal section 108, and the cannula 104 may define a lumen 110 extending therebetween. The proximal section 106 of the cannula 104 may be coupled (e.g., releasably coupled) to the hub 102, and the hub 102 may be graspable for single-handed operation by a clinician to deliver the cannula 104 to the treatment site, with such single-handed delivery of the cannula 104 to the treatment site being similar to typical needle delivery and, thus, familiar to clinicians. As described in greater detail below, the hub 102 may be manually actuatable to deliver the fastener assembly 200 from the cannula 104 to the treatment site.

In general, the cannula 104 may be sized for percutaneous delivery of the fastener assembly 200 to the treatment site. Thus, for example, the distal section 108 of the cannula 104 may be sharp (e.g., including one or more beveled edges) for puncturing skin of the subject and moving through biological tissue to the treatment site. Further, or instead, the cannula 104 may be substantially cylindrical and, in particular, may be sized according to any one or more of various different needle gauges suitable for, among other things, providing column strength suitable for resisting buckling as the cannula 104 punctures the skin of the subject and moves through the biological tissue to the treatment site.

In general, the first T-fastener 202, the second T-fastener 204, and at least a portion of the suture 206 may be at least partially disposed within the cannula 104. Given that there is generally at least some slack in the suture 206 as the first T-fastener 202 and the second T-fastener 204 are disposed in the cannula 104, the suture 206 may be managed to reduce the likelihood of tangling as the suture 206 is delivered from the cannula 104, along with the delivery of the first T-fastener 202 and the second T-fastener 204. For example, in certain implementations, the first end portion 208 and the second end portion 210 of the suture 206 may be disposed outside of the needle assembly 100. With the first end portion 208 and the second end portion 210 disposed outside of the needle assembly 100, a clinician may hold the first end portion 208 and the second end portion 210 of the suture 206, creating a small amount of tension in the length 212 of the suture 206 to reduce the likelihood of the suture 206 becoming tangled with itself or with one or more of the first T-fastener 202 or the second T-fastener 204.

The first T-fastener 202 and the second T-fastener 204 may be each be positionable in a first orientation in the lumen 110 defined by the cannula 104 such that the first longitudinal axis 209 defined by the first elongate body 214 and the second longitudinal axis 217 defined by second elongate body 216 are each substantially aligned with a cannula center axis 112 defined by the cannula 104. In this context, substantial alignment shall be understood to include any orientation in which the first T-fastener 202 and the second T-fastener 204 are each deliverable from the distal section of the cannula 104 in response to a force exerted by the hub 102 along the cannula center axis 112. Given this first orientation, it shall be appreciated that the appropriate sizing of the diameter of the lumen 110 relative to respective maximum radial dimensions of the first T-fastener 202 and the second T-fastener 204 may account for competing aspects of frictional forces. That is, at a minimum, the respective maximum radial dimensions of the first T-fastener 202 and the second T-fastener 204 may each be sized relative to the lumen 110 such that frictional force between the and each of the first T-fastener 202 and the second T-fastener 204 along the lumen 110 is at least sufficient to hold the first T-fastener 202 and the second T-fastener 204 substantially in place at a substantially fixed longitudinal position along the lumen 110 (e.g., during percutaneous delivery of the distal section 108 of the cannula 104 to the treatment site). At a maximum, however, the respective maximum radial dimensions of the first T-fastener 202 and the second T-fastener 204 may each be sized such that frictional force between the cannula 104 and each of the first T-fastener 202 and the second T-fastener 204 along the lumen 110 may be overcome by the force exerted by the hub 102 along the cannula center axis 112.

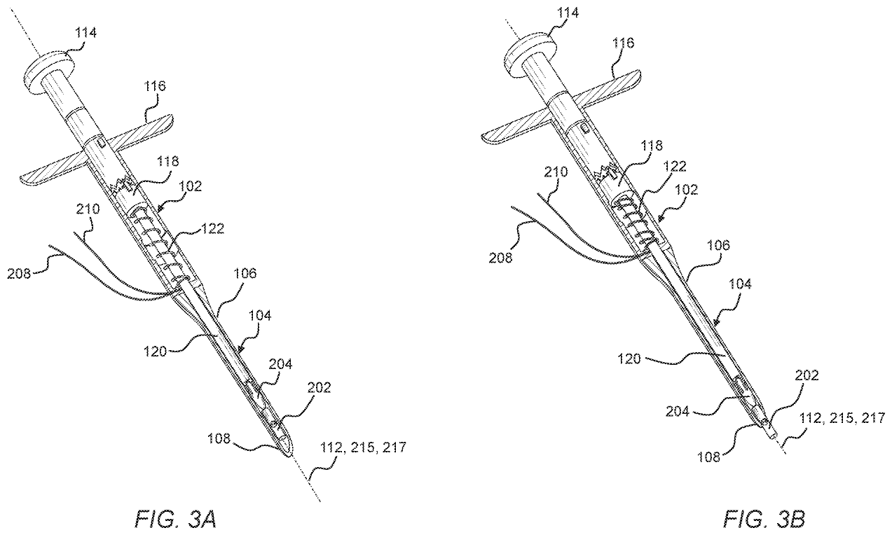

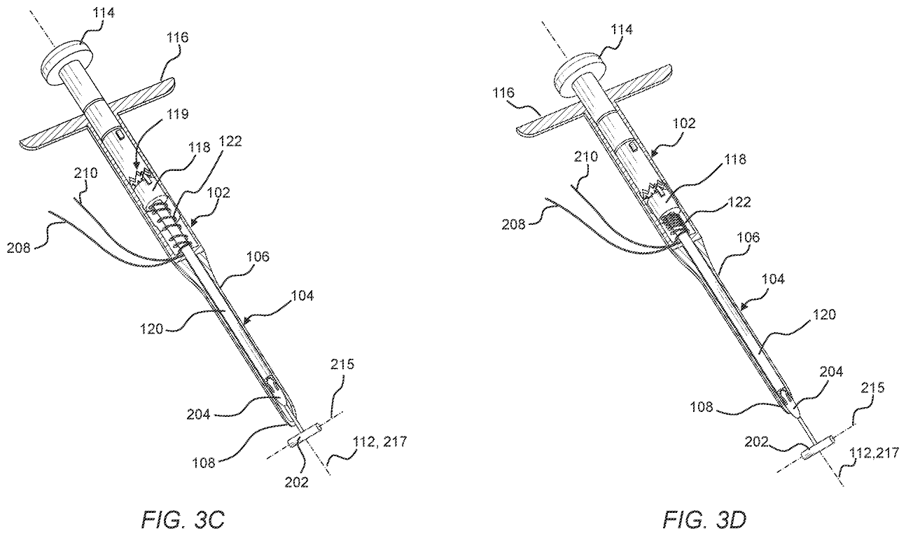

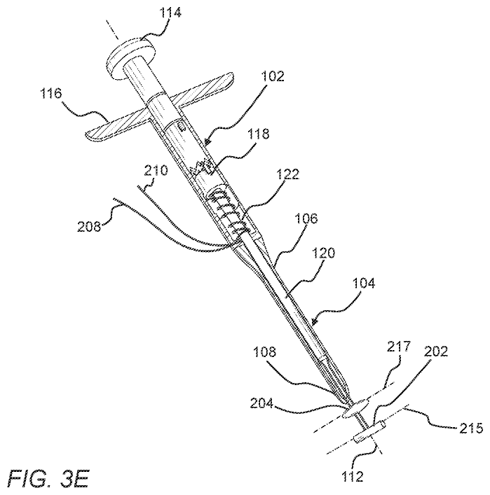

FIGS. 3A, 3B, 3C, 3D, and 3E are collectively a schematic representation of a temporal sequence of activation of the needle assembly 100 to deliver the fastener assembly 200. In particular, the temporal sequence shows the delivery of the fastener assembly 200 in discrete stages, in which the first T-fastener 202 may be delivered from the cannula 104 at a first anatomical position while the second T-fastener 204 remains in the cannula 104 and the second T-fastener 204 may be delivered from the cannula 104 in a controlled manner at a second anatomical position apart from the first anatomical position. The discreteness of the stages is useful, for example, for reducing the likelihood of inadvertently delivering one or both of the first T-fastener 202 or the second T-fastener 204 prematurely. That is, with appropriate tactile feedback from the hub 102, the clinician may discern a first discrete stage associated with delivery of the first T-fastener 202 from a second discrete stage associated with delivery of the second T-fastener 204. In this manner, the hub 102 may advantageously simplify sequential delivery of the two T-fasteners for a clinician by providing two separate user actions each of which discretely delivers one of the T-fasteners.

In general, the hub 102 may include a button 114, a handle 116, a cam 118, a plunger 120, and a spring 122. The button 114 may be proximal to the handle 116 and positioned relative to the handle 116 such that the button 114 may be depressible toward the distal section 108 of the cannula 104 by the clinician using single-handed operation (e.g., using a motion used to depress a plunger of a syringe). The button 114 may be slidably engageable with the cam 118 to facilitate moving the cam 118 in a distal direction parallel to the cannula center axis 112 while allowing the cam 118 to rotate about the cannula center axis 112. The cam 118 may be coupled (e.g., directly coupled) to the plunger 120, with the plunger 120 extending into the cannula 104. The cam 118 may be additionally, or alternatively, coupled (e.g., mechanically coupled) to the spring 122 to bias the cam 118 to move in the proximal direction following depression of the button 114 in the distal direction. Based on a combination of force exerted on the cam 118 in the proximal direction by depression of the button 114 and force exerted on the cam 118 in the distal direction through expansion of the spring 122, the cam 118 may move along grooves defined along an interior surface of the hub 102 to control movement of the plunger 120 in discrete stages, each having a linear position and a rotational orientation defined by a location of the cam 118 in one of the grooves, and the plunger 120 moving into different discrete stages with successive depressions of the button 114 as guided by the grooves. This process is described in greater detail below and may be initiated by a control surface 119 coupled to the button 114 that engages the cam 118 and applies and axial and rotational force to the cam 118 as the button 114 is depressed in order to guide the cam 118 into the grooves. The controlled movement of the cam 118 along the path defined by the grooves may then facilitate staged delivery of the first T-fastener 202 and the second T-fastener 204 (at different axial positions of the plunger 120) while being substantially independent of force exerted on the button 114 above a minimum threshold.

Referring now FIGS. 3A and 3B, the button 114 may be depressed to move the plunger 120 in a first discrete movement. For example, the button 114 may be depressed a first time (e.g., a first click) in the distal direction to move the plunger 120 toward the distal section 108 of the cannula 104. The plunger 120 may be in contact with the second T-fastener 204 such that, as the plunger 120 is depressed in a first discrete movement in the distal direction in the cannula 104, the first discrete movement of the plunger 120 in the distal direction moves the first T-fastener 202, the second T-fastener 204, and at least a portion of the length 212 of the suture 206 in the distal direction along the lumen 110. In particular, via the first discrete movement of the plunger 120 in the distal direction, the force exerted by the plunger 120 on the second T-fastener 204 may move and, ultimately, deliver the first T-fastener 202 and at least a portion of the length 121 of the suture 206 from the distal section 108 of the cannula 104.

In certain instances, as the plunger 120 moves in the distal direction from a first, original position to a second position and deliver the first T-fastener 202 beyond the distal section 108 of the cannula 104. As described in greater detail below, the spring 122 may return the plunger 120 partially from the second position to a third position that is between the second position and the first, original position of the plunger 120. That is, the spring 122 may move the plunger 120 in a proximal direction between the first and second discrete movements associated with delivering the first T-fastener 202 and the second T-fastener 204. More specifically, the spring 122 may move the plunger 120 and, thus, the button 114 in the proximal direction to provide feedback to the clinician (e.g., a click, tactile feedback, or a combination thereof) following the first discrete movement of the plunger 120. For example, the feedback may indicate to the clinician that the first T-fastener 202 has been properly delivered from the cannula 104 and, thus, based on such feedback, the clinician may retract the cannula 104 to a position suitable for delivery of the second T-fastener 204.

Referring now to FIGS. 3B and 3C, following delivery of the first T-fastener 202 from the cannula 104, the second T-fastener 204 may remain in the cannula 104 with the second longitudinal axis 217 of the second T-fastener 204 substantially aligned with the cannula center axis 112 to facilitate moving the second T-fastener 204 to a second anatomical location by retracting the cannula 104. With the second T-fastener 204 in the cannula 104, the first T-fastener 202 may pivot relative to the cannula 104 such that the first longitudinal axis 215 of the first T-fastener 202 may intersect the second longitudinal axis 217 of the second T-fastener 204. Such pivotability of the first T-fastener 202 may facilitate anchoring the first T-fastener 202 in place at a first anatomical location as the cannula 104 is retracted to the second anatomical location. For example, as the first longitudinal axis 215 of the first T-fastener 202 pivots to be transverse to the second longitudinal axis 217 of the second T-fastener 204 disposed in the cannula 104, the first T-fastener 202 may be less likely to retract inadvertently back through the puncture made by the cannula 104 in the biological tissue.

Referring now to FIGS. 3C and 3D, the button 114 may be depressed a second time to move the plunger 120 in a second discrete movement in the distal direction of the cannula 104. For example, the second discrete movement of the plunger 120 may be different from the first discrete movement of the plunger 120. As a more specific example, the second discrete movement of the plunger in the distal direction may deliver the second T-fastener 204 from the distal section 108 of the cannula 104. In certain implementations, as the plunger 120 moves according to the second discrete movement, the plunger 120 may move from the third position to a fourth position as the second T-fastener 204 is delivered from the distal section 108 of the cannula 104. In certain instances, the fourth position of the spring 122 may be more compressed than the second position of the spring 122.

Referring now to FIGS. 3D and 3E, following the second discrete movement of the plunger 120, the spring 122 may move the plunger 120 in the proximal direction from the fourth position back to the first, original position. Such movement of the plunger 120 may provide the clinician with feedback (e.g., a click, tactile feedback, or a combination thereof) indicating that the second T-fastener 204 and, thus, the fastener assembly 200 has been delivered from the cannula 104. Based on this feedback, the clinician may withdraw the cannula 104 from the subject, as described in greater detail below.

Following delivery from the distal section 108 of the cannula 104, the second T-fastener 204 may pivot relative to the cannula 104 such that the second longitudinal axis 217 of the second T-fastener 204 may intersect the cannula center axis 112. Such pivotability of the second T-fastener 204 may facilitate anchoring the second T-fastener 204 in place at a second anatomical location as the cannula 104 is retracted from the subject. As an example, with the second longitudinal axis 217 of the second T-fastener 204 transverse to the cannula center axis 112, the second T-fastener 204 may be less likely to retract inadvertently back through the puncture made by the cannula 104 in the biological tissue.

Referring now to FIGS. 1D and 3A-3E, the movement of the plunger 120 between the positions associated with the discrete delivery of the first T-fastener 202 may be controlled to provide the clinician feedback regarding successful delivery of the first T-fastener 202. In general, the plunger 120 may move between the positions associated with the discrete delivery of the first T-fastener 202 and the second T-fastener 204 through controlled movement along a path 130 (e.g., a stepped helical path) defined by an inner wall 132 of the hub 102. For example, the spring 122 may bias the cam 118 against a first edge 134 at least partially defining the path 130. As the cam 118 rotates along the first edge 134 of the path 130, the longitudinal position of the cam 118 and, thus, the longitudinal position of the plunger 120 may change. In particular, the movement of the cam 118 along the first edge 134 may move the plunger 120 through the predetermined series from the first position to the second position (the first discrete movement shown in FIGS. 3A and 3B), from the second position to the third position to provide feedback (FIGS. 3B and 3C), from the third position to the fourth position (the second discrete movement shown in FIGS. 3C and 3D), and from the fourth position back to the first, original position to provide feedback (FIGS. 3D and 3E).

In certain implementations, the path 130 may be further, or instead, defined by a second edge 136 that limits the distal movement of the cam 118. That is, the second edge 136 may limit distal movement of the cam 118 and, thus, the plunger 120 such that the discrete delivery of the first T-fastener 202 and then the second T-fastener 204 is substantially independent of the amount of force used to depress the button 114 each time. For example, in the event that a large amount of force is used to depress the button 114 the first time, the second edge 136 may limit the amount of distal travel of the plunger 120 to a point sufficient to deliver the first T-fastener 202 from the distal section 108 of the cannula 104 while the second T-fastener 204 remains in the cannula 104. Accordingly, by limiting the distal travel of the plunger 120 in response to large actuation forces, the second edge 136 may reduce the likelihood of prematurely delivering the second T-fastener 204 from the distal section 108 of the cannula 104 in response to a range of forces that may be encountered in use.

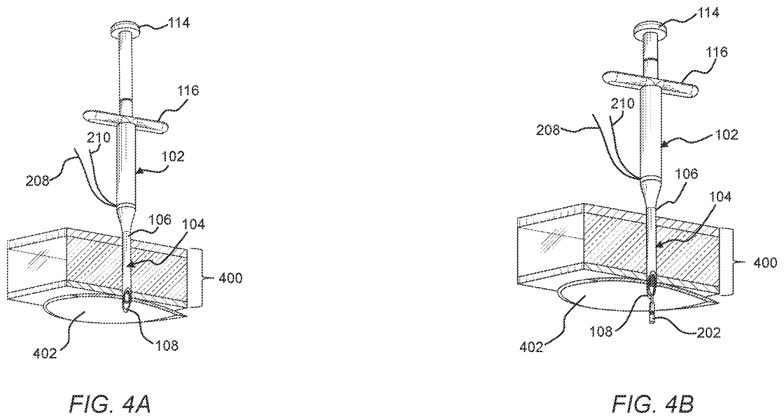

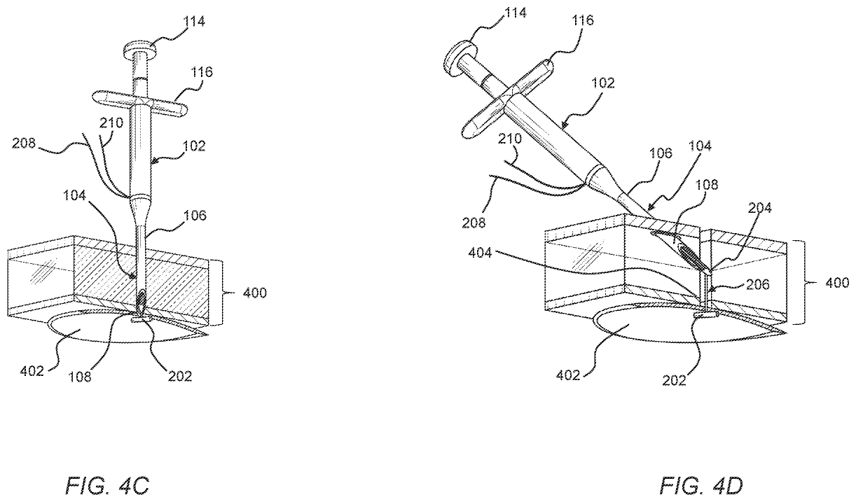

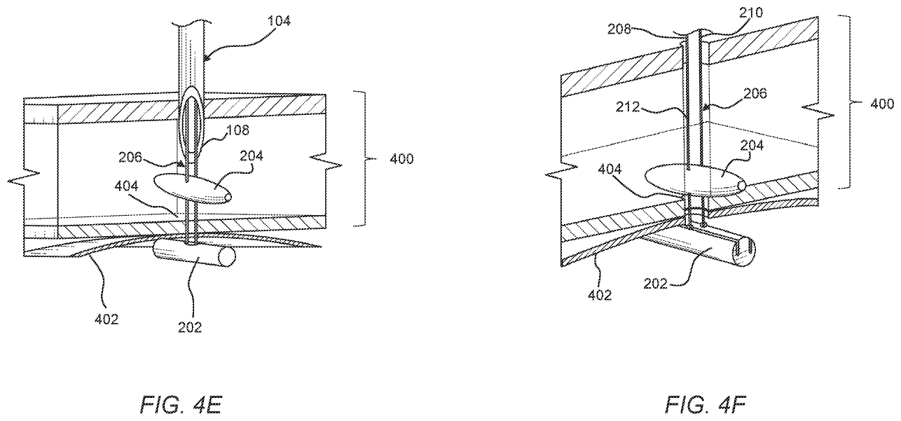

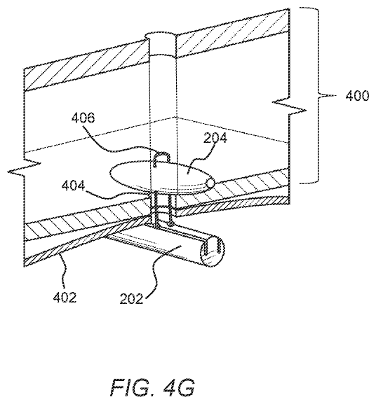

FIGS. 4A-4G are collectively a schematic representation of a temporal sequence of an exemplary method of intracorporeal delivery of a fastener assembly to a mammal. For the sake of clarity of explanation, the exemplary method is described with respect to intracorporeal delivery of the fastener assembly 200 using the needle assembly 100. However, unless otherwise specified or made clear from the context, it shall be understood that any of the various different aspects of the exemplary method may be carried out using any one or more of the various different fastener assemblies and needle assemblies described herein.

Referring now to FIG. 4A, the distal section 108 of the cannula 104 may be moved at least partially through biological tissue 400 to a first anatomical location 402. For example, moving the distal section 108 of the cannula 104 through the biological tissue 400 may include puncturing the biological tissue 400 with one or more sharp edges of the distal section 108 and moving the distal section 108 percutaneously to the first anatomical location 402. In general, the biological tissue 400 may be any of various different types of biological tissue 400 associated with a given procedure. Thus, for example, the biological tissue 400 may include a plurality of layers of tissue. As a more specific example, the biological tissue 400 may include the stomach wall and the abdominal wall of the subject in instances in which the exemplary method is carried out as part of a G-tube placement procedure. Additionally, or alternatively, moving the distal section 108 of the cannula 104 through the biological tissue 400 may include moving the distal section 108 of the cannula 104 through one or more implants (e.g., synthetic implants).

Referring now to FIGS. 4B and 4C, with the distal section 108 of the cannula 104 at the first anatomical location 402, the first T-fastener 202 may be delivered from the cannula 104 to the first anatomical location 402. In general, the first T-fastener 202 may be delivered from the distal section 108 of the cannula 104 according to any one or more of the various different techniques described herein. Thus, for example, delivering the first T-fastener 202 may include depressing the button 114 to move the first T-fastener 202 from the distal section of the cannula 104. Further, or instead, at the first anatomical location 402, the first T-fastener 202 may become oriented transverse to the direction of the puncture made by the cannula 104 such that the first T-fastener 202 may resist movement back through the biological tissue 400 and, thus, remains substantially in place at the first anatomical location 402.

Referring now to FIG. 4D, with the first T-fastener 202 at the first anatomical location 402, the distal section 108 of the cannula 104 may be retracted through the biological tissue 400 to a second anatomical location 404. For example, the second anatomical location 404 may be proximal to the first anatomical location 402 along the path of the puncture made by the distal section 108 of the cannula 104 through the biological tissue 400. In certain instances, retracting the distal section 108 of the cannula 104 may include tilting the cannula 104 to position the cannula 104 for subsequent delivery of the second T-fastener 204. Further, or instead, in instances in which the biological tissue 400 includes multiple layers, it shall be appreciated that the second anatomical location 404 may be spaced from the first anatomical location 402 by the plurality of layers.

Referring now to FIGS. 4D and 4E, the second T-fastener 204 may be delivered from the distal section 108 of the cannula 104 to the second anatomical location 404. In general, the second T-fastener 204 may be delivered to the second anatomical location 404 according to any one or more of the delivery techniques described herein. Thus, for example, delivery of the second T-fastener 204 may include a second actuation of the button 114. Further, or instead, in instances in which the cannula 104 is tilted as the second T-fastener 204 is delivered to the second anatomical location 404, the tilting may facilitate orienting the second T-fastener 204 transverse to a direction of the puncture made by the distal section 108 of the cannula 104. Such a transverse orientation of the second T-fastener 204 relative to the direction of puncture may reduce the likelihood of inadvertent migration of the second T-fastener 204 back through the puncture in the biological tissue 400 before the second T-fastener 204 and the first T-fastener 202 are secured to one another.

Referring now to FIG. 4F, the first T-fastener 202 and the second T-fastener 204 may be moved toward one another along the suture 206 extending through the first T-fastener 202 and the second T-fastener 204. For example, with the suture 206 pulled taught to exert a proximal force on the first T-fastener 202, the second T-fastener 204 may be pushed (e.g., through a portion of a knot being pulled taut) in a distal direction toward the first T-fastener 202. In general, the movement of the first T-fastener 202 and the second T-fastener 204 toward one another along the suture 206 may secure the biological tissue 400 therebetween, as may be an appropriate for a given medical procedure.

Referring now to FIG. 4G, a knot 406 may be formed in the suture 206 to hold the first T-fastener 202 and the second T-fastener 204 in place. In certain implementations, the knot 406 may be subcutaneous such that, following the procedure, the fastening is entirely internal to the subject. This may be useful, for example, for reducing the likelihood of infection or other post-operative complications.

While certain implementations have been described, other implementations are additionally or alternatively possible.

For example, while first T-fasteners and second T-fasteners have each been described as having certain features, other features are additionally or alternatively possible. As an example, the first T-fastener may be identical to the second T-fastener in some implementations. More generally, each of the first T-fastener or the second T-fastener may have any one or more of the features of the other one of the first T-fastener or the second T-fastener, unless otherwise specified or made clear from the context.

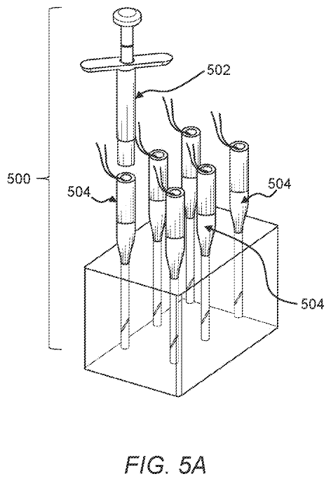

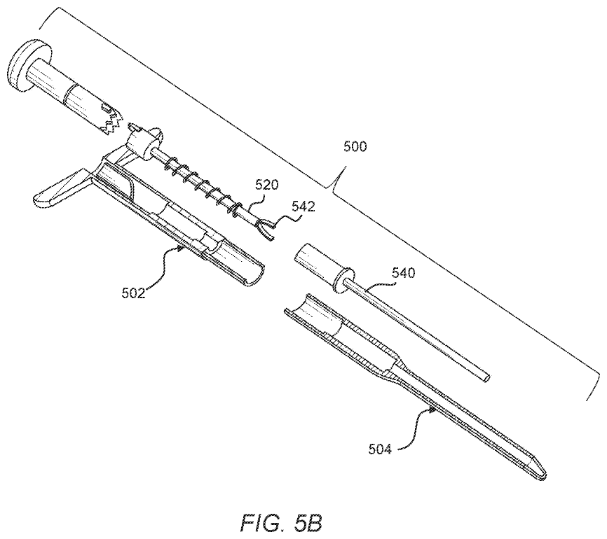

As another example, while needle assemblies have generally been described as including a hub coupled to a cannula, other arrangements are additionally or alternatively possible. For example, referring now to FIGS. 5A and 5B, a needle assembly 500 may include a hub 502 and a cannula 504 releasably securable (e.g., using an interference fit, a threaded engagement, a snap fit, or a combination thereof) to the hub 502. Unless otherwise indicated, elements designated with 500-series element numbers in FIGS. 5A and 5B shall be understood to be similar to corresponding elements designated with 100-series element in the other figures and, therefore, are not described separately, except to highlight differences or to emphasize certain aspects. As one example, the hub 502 shall be understood to correspond to the hub 102 (FIG. 1A), unless otherwise specified.

Referring now to FIG. 5A, multiple instances of the cannula 504 may be pre-loaded with respective fastening assemblies (e.g., the fastener assembly 200 in FIG. 2A). A clinician may couple the hub 502 to a first instance of the cannula 504 preloaded with a fastener assembly. The clinician may then deliver the fastener assembly to a treatment site according to any one or more of the methods described herein. Once the fastener assembly is delivered and the first instance of the cannula 504 is removed from the subject, the hub 502 may be decoupled from the first instance of the cannula 504. The hub 502 may then be coupled to a second instance of the cannula 504 preloaded with a fastener assembly, and the process may be repeated as necessary to deliver a number of fastening assemblies to the treatment site. That is, stated differently, through releasable engagement of the hub 502 and the cannula 504, the hub 502 may be reused to save cost without significantly delaying in the overall procedure. Further, or instead, the releasable engagement of the hub 502 and the cannula 504 may facilitate sterilization. For example, the hub 502 and the cannula 504 may be sterilized separately, which may be useful for using different types of material in the hub 502 and the cannula 504 (and, in some instances, a fastener assembly preloaded in the cannula 504).

Referring now to FIG. 5B, the needle assembly 500 may, additionally or alternatively, include a plunger 520 and a casing 540. The plunger 520 may be disposed in and coupled to the hub 502. Further, or instead, the casing 540 may be disposed in and coupled to the cannula 504. The plunger 520 may include a ratchet 542 releasably securable to the casing 540 such that, in use, the hub 502 and the cannula 504 may be releasably securable to one another at least partially through releasable engagement of the plunger 520 to the casing 540. It shall be appreciated that the plunger 520 and the casing 540 releasably secured to one another may cooperate to function as the plunger 120 (FIG. 1B) to deliver the fastener assembly 200 (FIG. 2A) from the cannula 504 via actuation of the hub 502.

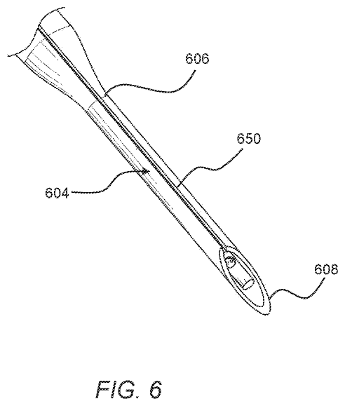

As still another example, while cannulas have generally been described as having continuous circumferences such that ends of sutures move through the lumen of the cannula as the cannula is retracted, other types of circumferences are additionally or alternatively possible. For example, referring now to FIGS. 2A and 6, a cannula 604 may have a proximal section 606 and a distal section 608. The cannula 604 may define a slit 650 extending from the proximal section 606 to the distal section 608. In general, the slit 650 may have a cross-sectional dimension larger than a radial dimension of the suture 206. The length 212 of the suture 206 may extend through the slit 650 with the first end portion 208 and the second end portion 210 each extending outside of the cannula 604. In use, the suture 206 may slide along the slit 650 as the fastener assembly 200 is delivered from the cannula 604. That is, the slit 650 may control the position of the suture 206 during delivery of the fastener assembly 200. This may, for example, reduce the likelihood of tangling of the suture 206 as the suture is delivered. Additionally, or alternatively, the slit 650 may be positioned away from one or more sharp edges of the distal section 608 to reduce the likelihood of inadvertently cutting the suture 206 on the one or more sharp edges.

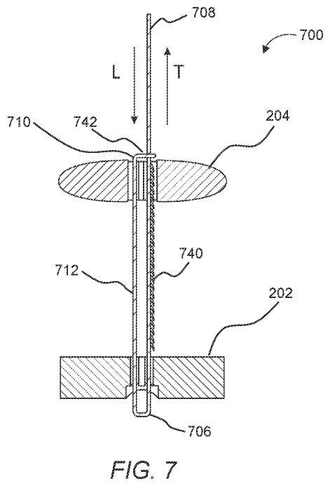

As still another example, while fastener assemblies have been described as including sutures that may be filaments, other types of sutures are additionally or alternatively possible. For example, referring now to FIG. 7, a fastener assembly 700 may include the first T-fastener 202, the second T-fastener 204, and a suture 706 extending through the first T-fastener 202 and the second T-fastener 204. Unless otherwise indicated, elements designated with 700-series element numbers in FIG. 7 shall be understood to be similar to corresponding elements designated with 200-series element in the other figures and, therefore, are not described separately, except to highlight differences or to emphasize certain aspects. Thus, the suture 706 shall be understood to correspond to the suture 206 (FIG. 2A), unless otherwise specified.

In general, the suture 706 may include a first end portion 708, a second end portion 710, and a length 712 extending therebetween. The suture 706 may include a plurality of barbs 740 extending radially from the length 712 of the suture 706. In general, at least one of the first end portion 708 or the second end portion 710 of the suture 706 may be movable through the plurality of second openings 220 of the second T-fastener in a first direction “T” to move at least one of the barbs 740 through at least one opening of the plurality of second openings 220. In certain implementations, the at least one opening of the plurality of second openings 220 may be sized to resist movement of the length 712 of the suture 706 through the at least one opening of the plurality of second openings 220 in a second direction “L” opposite the first direction “T.” Additionally, or alternatively, the suture 706 may include a loop 742 on the second end portion 710 of the suture 706. The loop 742 may be sized to resist movement of the length 712 of the suture 706 through the at least one opening of the plurality of second openings 220 in the second direction “L” opposite the first direction “T.” Thus, in general, the barbs 740 may facilitate securing biological tissue between the first T-fastener 202 and the second T-fastener 204 without the need to tie a knot in the suture 706. Accordingly, as compared to implementations requiring the use of a knot, the suture 706 may reduce the time and skill required to secure biological tissue between the first T-fastener 202 and the second T-fastener 204.

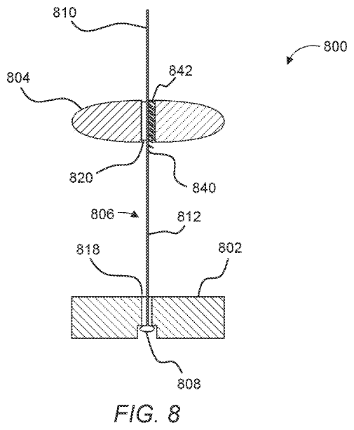

As yet another example, while a barbed suture has been described as being engageable with a loop in the suture to restrict movement of the fastener, other types of engagement of the barb to restrict movement of the fastener are additionally or alternatively possible. For example, referring now to FIG. 8, a fastener assembly 800 may include the first T-fastener 802, the second T-fastener 804, and a suture 806 extending through the first T-fastener 802 and the second T-fastener 804. Unless otherwise indicated, elements designated with 800-series element numbers in FIG. 8 shall be understood to be similar to corresponding elements designated with 200-series element in the other figures and, therefore, are not described separately, except to highlight differences or to emphasize certain aspects. Thus, the suture 806 shall be understood to correspond to the suture 206 (FIG. 2A), unless otherwise specified.

In general, the suture 806 may include a first end portion 808, a second end portion 810, and a length 812 extending therebetween. The suture 806 may include a plurality of barbs 840 extending radially from the length 812 of the suture 806. The first end portion 808 of the suture 806 may be larger than a first opening 818 defined by the first T-fastener 802 such that the first T-fastener 802 is restricted from moving past the first end portion 808 of the suture 806. The second T-fastener 804 may define a second opening 820, and at least one pawl 842 extending into the second opening 820. As the second end portion 810 of the suture 806 is pulled in a direction away from the first T-fastener 802, the barbs 840 of the suture 806 may engage the at least one pawl 842 in the second opening 820. In particular, the barbs 840 and the at least one pawl 842 may be oriented relative to one another such that the suture 806 is movable through the second opening 820 only in the direction away from the first T-fastener 802. That is, the suture 806 may be movable through the second opening 820 only to decrease a relative distance between the first T-fastener 802 and the second T-fastener 804, thus securing biological tissue between the first T-fastener 802 and the second T-fastener 804. In general, the at least one pawl 842 may restrict (e.g., prevent) movement of the suture 806 in a direction toward the first T-fastener 802 such that biological tissue may be secured between the first T-fastener 802 and the second T-fastener 804 without the use of a knot.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the devices, systems, and methods described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A system for intracorporeal delivery of a fastener assembly, the system comprising:
a needle assembly including a hub for single-handed operation by a clinician and a cannula coupled at a proximal section to the hub, the cannula having a distal section for minimally invasive delivery to a treatment site, and the cannula defining a lumen extending from the proximal section to the distal section,
a first T-fastener having a first elongate body, the first T-fastener disposed within the lumen and having a first longitudinal axis aligned to an axis of the lumen;
a second T-fastener having a second elongate body, the second T-fastener disposed within the lumen and having a second longitudinal axis aligned to the axis of the lumen and the first longitudinal axis of the first T-fastener, and the second T-fastener closer to the hub than the first T-fastener along the axis of the lumen;
a suture having a first end portion, a second end portion, and a length therebetween, at least a portion of the length disposed in the lumen and extending through a plurality of openings in the first T-fastener and the second T-fastener; and
a plunger operable from the hub to separately and sequentially deploy the first T-fastener and the second T-fastener beyond the distal section of the lumen at the treatment site.

2. A fastener assembly for intracorporeal tissue apposition, the fastener assembly comprising:
a first T-fastener having a first elongate body defining a plurality of first openings;
a second T-fastener having a second elongate body defining a plurality of second openings; and
a suture having a first end portion, a second end portion, and a length therebetween, the length of the suture extending through the plurality of first openings and the plurality of second openings with the second T-fastener, along the length of the suture, between the first T-fastener and each of the first end portion and the second end portion of the suture.

3. The fastener assembly of claim 2, wherein at least the second T-fastener is movable along the length of the suture in a direction toward the first T-fastener.

4. The fastener assembly of claim 3, wherein the first T-fastener is secured in a fixed position along the length of the suture.

5. The fastener assembly of claim 2, wherein at least a portion of one or more of the first elongate body of the first T-fastener, the second elongate body of the second T-fastener, or the suture is bioabsorbable.

6. The fastener assembly of claim 2, wherein each first opening of the plurality of first openings defines a respective first center axis, each second opening of the plurality of second openings defines a respective second center axis, and, with tension in the length of the suture, each first center axis associated with the plurality of first openings of the first T-fastener is movable into substantial coaxial alignment with a respective second center axis associated with the plurality of second openings of the second T-fastener.

7. The fastener assembly of claim 6, wherein the first center axis defined by the plurality of first openings are substantially parallel to one another and spaced relative to one another by a first distance, and the second center axis defined by the plurality of second openings are substantially parallel to one another and spaced relative to one another by a second distance about equal to the first distance.

8. The fastener assembly of claim 6, wherein the first center axis defines a first plane substantially perpendicular to a first longitudinal axis defined by the first elongate body of the first T-fastener.

9. The fastener assembly of claim 6, wherein the second center axes define a second plane substantially perpendicular to a second longitudinal axis defined by the second elongate body of the second T-fastener.

10. The fastener assembly of claim 9, wherein the second elongate body of the second T-fastener has a maximum radial dimension along the second plane and, in each direction away from the second plane along the second longitudinal axis, the second elongate body of the second T-fastener has a decreasing radial dimension.

11. The fastener assembly of claim 2, wherein the first elongate body of the first T-fastener defines a pair of elongate grooves extending longitudinally along a longitudinal dimension of the first elongate body, and a portion of the length of the suture is positionable in the pair of elongate grooves.

12. The fastener assembly of claim 2, wherein the suture further includes a plurality of barbs, each barb of the plurality of barbs extends radially from the length of the suture, and at least one of the first end portion of the suture or the second end portion of the suture is movable through the plurality of second openings of the second T-fastener in a first direction to move at least one barb of the plurality of barbs through at least one opening of the plurality of second openings.

13. A system for intracorporeal delivery of a fastener assembly, the system comprising:
a first T-fastener having a first elongate body;
a second T-fastener having a second elongate body;
a suture having a first end portion, a second end portion, and a length therebetween, the length of the suture extending through a plurality of first openings defined by the first elongate body and through a second plurality of openings defined by the second elongate body; and
a needle assembly including a hub and a cannula, the cannula having a proximal section and a distal section, the hub coupled to the proximal section of the cannula, the cannula defining a lumen extending from the proximal section to the distal section, and each of the first T-fastener, the second T-fastener, and at least a portion of the length of the suture disposed in the lumen.

14. The system of claim 13, wherein the first elongate body defines a first longitudinal axis, the second elongate body defines a second longitudinal axis, and the cannula defines a cannula center axis substantially aligned with the first longitudinal axis and the second longitudinal axis.

15. The system of claim 14, wherein, with the second longitudinal axis of the second T-fastener substantially aligned with the cannula center axis in the lumen, the first T-fastener is positionable beyond the distal section of the cannula with the first longitudinal axis of the first T-fastener intersecting the second longitudinal axis of the second T-fastener.

16. The system of claim 13, wherein the first end portion and the second end portion of the suture are disposed outside of the needle assembly.

17. The system of claim 16, wherein the cannula defines a slit extending from the proximal section to the distal section, the slit has a cross-sectional dimension larger than a radial dimension of the suture, and the length of the suture extends through the slit with the first end portion and the second end portion of the suture outside of the cannula.

18. The system of claim 13, further comprising a plunger in contact with the second T-fastener, the plunger is depressible in a first discrete movement in a distal direction in the cannula, and the first discrete movement of the plunger in the distal direction moves the first T-fastener, the second T-fastener, and at least a portion of the length of the suture in the distal direction along the lumen.

19. The system of claim 18, wherein the plunger is depressible in a second discrete movement in the distal direction in the cannula, the first discrete movement of the plunger in the distal direction delivering the first T-fastener from the distal section of the cannula, and the second discrete movement of the plunger in the distal direction delivering the second T-fastener from the distal section of the cannula.

20. The system of claim 19, wherein, between the first discrete movement and the second discrete movement in the distal direction in the cannula, the plunger is movable in a proximal direction in the cannula.
